(19) United States
(12) Patent Application Publication
(10) Pub. No.: US 2012/0015938 A1
(43) Pub. Date: Jan. 19, 2012

(54) 1,4-BENZODIAZEPIN-2-ON DERIVATIVES

(75) Inventors: Masanori Asai, Ibaraki (JP); Tasuku Haketa, Hyogo (JP); Seiichi Inamura, Kanagawa (JP); Hideki Kurihara, Kanagawa (JP); Teruyuki Nishimura, Shizuoka (JP); Tadashi Shimamura, Ibaraki (JP)

(73) Assignee: MSD K.K., Tokyo (JP)

(21) Appl. No.: 13/144,090

(22) PCT Filed: Feb. 12, 2010
(86) PCT No.: PCT/JP2010/053178

§ 371 Date: Jul. 12, 2011

(30) Foreign Application Priority Data
Feb. 17, 2009 (JP) 2009-033877

(51) Int. Cl. A61K 31/5513; A61P 3/10; A61P 3/06; C07D 243/14; A61P 3/04

(52) U.S. Cl. 514/221; 540/509

(57) ABSTRACT

The present invention relates to a compound represented by a formula (I): wherein R¹ represents a halogen atom or the like; R² represents a hydrogen atom or the like; R³ and R⁴ each independently represents lower alkyl; R⁵ represents phenyl or the like; R⁶ represents a halogen atom or the like; m is an integer of from 0 to 2; p is an integer of from 1 to 4; and q is an integer of from 1 to 5 as an inhibitor of DGAT1 and its use for the treatment of hyperlipidemia etc.

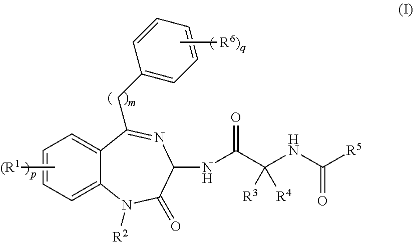

### TECHNICAL FIELD

The present invention relates to 1,4-benzodiazepin-2-on derivatives which are useful in the pharmaceutical field. These compounds have inhibitory activity of diacylglycerol O-acyltransferase type 1 (hereinafter also referred to as “DGAT1”) and are useful as agents for treating and/or preventing hyperlipidemia, diabetes and obesity.

### BACKGROUND ART

Obesity is a condition, in which the background of lack of exercise, intake of excessive energy, ageing, etc. leads to energy imbalance, the surplus energy is accumulated generally as neutral fat (triacylglycerol, TG) in adipose tissue, and a body weight and a fat mass are thus increased. In recent years, the concept of metabolic syndrome associated with obesity involving the accumulation of the visceral fat, as an upstream risk factor including a plurality of risk factors of diabetes, lipidosis, hypertension, etc. has been established, and the diagnostic criteria and therapeutic guidelines for the metabolic syndrome were formulated (Journal of Japan Society for the Study of Obesity, Vol. 12, Extra Edition, 2006). Since the metabolic syndrome results in increase in the risks of arteriosclerosis, cardiovascular disorder and cerebrovascular disorder, treatment of obesity has been recognized to be important for preventing these diseases.

Although the need of treating obesity is recognized to be important, there are extremely-limited drug therapies for obesity that are currently available, and the advent of novel antiobestic drugs having more definite action and few side-effects is thus desired.

In the living body, there are two TG synthesis pathways of a glycerol phosphate pathway, which is present in most organs and causes de novo TG synthesis, and a monoacylglycerol pathway, which is involved principally in absorption of aliphatic acid from the small intestine. Diacylglycerol acyltransferases (DGATs, EC 2.3.1.20), which are membrane-bound enzymes present in the endoplasmic reticulum, catalyze the final step of the TG synthesis common to the two TG synthesis pathways, that is, the reaction of transferring an acyl group of acyl-coenzyme A to the 3-position of 1,2-diacylglycerol to generate TG (Prog. Lipid Res., 43, 134-176, 2004; Ann. Med., 36, 252-261, 2004). DGATs have been found to include two subtypes of DGATs 1 and 2. There is no significant homology at the generic or amino acid level between the DGATs 1 and 2, which are encoded by different genes (Proc. Natl. Acad. Sci. USA., 95, 13018-13023, 1998; JBC, 276, 38870-38876, 2001). DGAT1, which is present in the small intestine, adipose tissue, the liver, etc., is believed to be involved in lipid absorption; lipid accumulation in the fat cell; and VLDL secretion and lipid accumulation in the liver, in the small intestine, the fat cell and the liver, respectively (Ann. Med., 36, 252-261, 2004; JBC, 280, 21506-21514, 2005). In consideration of these functions of DGAT1, a DGAT1 inhibitor is expected to improve metabolic syndrome through inhibition of the lipid absorption in the small intestine, the lipid accumulation in the adipose tissue and the liver, and the lipid secretion from the liver.

In order to carry out in vivo examination of the physiological function(s) of DGAT1 and inhibitory activity against DGAT 1, DGAT1-knockout mice deficient in DGAT1 at the generic level were produced, and analyses thereof were conducted. As a result, the DGAT1-knockout mice have been found to have smaller fat masses than those of wild-type mice and to exhibit resistance to obesity, abnormal glucose tolerance, insulin resistance and fatty liver due to a high-fat diet load (Nature Genetics, 25, 87-90, 2000; JCI, 109, 1049-1055, 2002). In addition, energy expense has been reported to be accelerated in the DGAT1-knockout mice; and transplantation of the adipose tissues of DGAT1-knockout mice into wild-type mice has been reported to make the wild-type mice resistant to obesity and abnormal glucose tolerance, induced by a high-fat diet load (JCI, 111, 1715-1722, 2003; Diabetes, 53, 1445-1451, 2004). In contrast, obesity and diabetes due to a high-fat diet load have been reported to worsen in mice with overexpression of DGAT1 in adipose tissue (Diabetes, 51, 3189-3195, 2002; Diabetes, 54, 3379-3386).

From the results, DGAT1 inhibitors are likely to be therapeutic drugs with efficacy for obesity or type 2 diabetes, lipidosis, hypertension, fatty liver, arteriosclerosis, cerebrovascular disorder, coronary artery disease, or the like, associated with the obesity.

Some compounds having DGAT1 inhibitory activity have been known, all of which have different structures from that of a compound according to the present invention (for example, see WO 2004/100881, WO 2006/044775 and WO 2006/113919).

Compounds having 1,4-benzodiazepin-2-on derivatives, which are disclosed in U.S. Pat. No. 6,759,404, also have different structures from that of a compound according to the present invention. Furthermore, although the U.S. patent discloses that the compounds disclosed therein have the action of inhibiting generation of an Aβ peptide and thereby preventing formation of amyloid protein deposited in the nerve, it does not disclose or suggest that the 1,4-benzodiazepin-2-on derivatives are useful in treatment and/or prevention of hyperlipidemia, diabetes and obesity.

### DISCLOSURE OF THE INVENTION

It is desirable to provide 1,4-benzodiazepin-2-on derivatives having DGAT1 inhibitory activity.

The present inventors conducted extensive research for developing a compound having DGAT1 inhibitory activity, found that a compound according to the present invention is efficacious as the compound having the DGAT1 inhibitory activity, and thus accomplished the present invention based on such findings.

Specifically, the present invention relates to a compound represented by a formula (I):

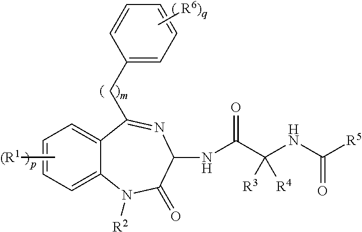

wherein R¹ each independently represents a hydrogen atom, a halogen atom or trifluoromethoxy;
R² represents lower alkyl, which may be substituted with 1 to 3 same or different halogen atoms;
R³ and R⁴ each independently represent lower alkyl;
R⁵ is a group selected from the group consisting of:
(1) phenyl, which may be substituted with 1 to 3 same or different groups selected from the group consisting of halogen atoms, lower alkoxy and trifluoromethyl;
(2) heteroaryl selected from the group consisting of pyridinyl, pyrazinyl, imidazolyl, thiazolyl, thienyl and oxazolyl, which heteroaryl may be substituted with 1 to 3 same or different halogen atoms or lower alkyl groups which may be substituted with 1 to 3 same or different halogen atoms;
(3) —O—C₃₋₆ branched lower alkyl, which may be substituted with 1 to 3 same or different halogen atoms; and
(4) C₃₋₇ cycloalkyl, which may be substituted with trifluoromethyl;
R⁶ each independently represents a group selected from the group consisting of a hydrogen atom, a halogen atom and trifluoromethoxy;
m is an integer of from 0 to 2;
p is an integer of from 1 to 4; and
q is an integer of from 1 to 5,
or a pharmaceutically acceptable salt thereof.

The present invention also relates to a pharmaceutical composition containing the compound represented by the formula (I) and a pharmaceutically acceptable carrier.

The present invention also relates to a DGAT1 inhibitor containing the compound represented by the formula (I) or a pharmaceutically acceptable salt thereof as an active ingredient.

The present invention also relates to an agent for treating and/or preventing hyperlipidemia, diabetes and obesity, which contains the compound represented by the formula (I) or a pharmaceutically acceptable salt thereof as an active ingredient.

A compound (1) according to the present invention or a pharmaceutically acceptable salt thereof has strong DGAT1 inhibitory activity and is thus useful for treating and/or preventing hyperlipidemia, diabetes and obesity.

The meanings of terms as used herein are described below, and a compound according to the present invention is described in further detail.

The term “halogen atom” encompasses, for example, fluorine chlorine, bromine and iodine atoms.

The term “lower alkyl” refers to linear or branched C₁₋₆ alkyl, examples of which include methyl, ethyl, propyl, isopropyl, butyl, isobutyl, sec-butyl, tert-butyl, pentyl, isoamyl, neopentyl, isopentyl, 1,1-dimethylpropyl, 1-methyl butyl, 2-methyl butyl, 1,2-dimethylpropyl, hexyl, isohexyl, 1-methyl pentyl, 2-methyl pentyl, 3-methyl pentyl, 1,1-dimethyl butyl, 1,2-dimethyl butyl, 2,2-dimethyl butyl, 1,3-dimethyl butyl, 2,3-dimethyl butyl, 3,3-dimethyl butyl, 1-ethyl butyl, 2-ethyl butyl, 1,2,2-trimethylpropyl and 1-ethyl-2-methylpropyl.

The term “lower alkoxy” refers to a group in which the hydrogen atom of hydroxy is substituted with the above-mentioned lower alkyl, examples of which include methoxy, ethoxy, propoxy, isopropoxy, butoxy, sec-butoxy, tert-butoxy, pentyloxy, isopentyloxy, hexyloxy and isohexyloxy.

Examples of “C₃₋₆ branched lower alkyl” specifically include isopropyl, sec-butyl, tert-butyl and 1-ethylpropyl.

The term “C₃₋₇ cycloalkyl” refers to a cycloalkyl group composed of 3 to 7 carbon atoms and specifically encompasses cyclopropyl, cyclobutyl, cyclopentyl, cyclohexyl and cycloheptyl.

In order to further specifically disclose a compound according to the present invention, represented by the formula (I):

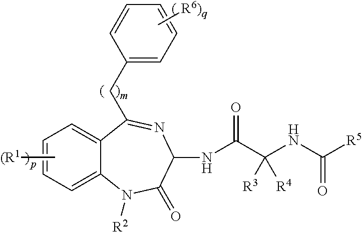

wherein each symbol has the same definition specified above, each symbol used in the formula (I) is described referring to specific examples.

R¹ each independently represents a hydrogen atom, a halogen atom or trifluoromethoxy.

“Halogen atom” represented by R¹ encompasses the same groups as the halogen atoms defined above, of which examples specifically include fluorine, chlorine, bromine and iodine atoms.

R² represents lower alkyl, which may be substituted with 1 to 3 same or different halogen atoms.

“Lower alkyl” represented by R² encompasses the same groups as the lower alkyl defined above, of which examples specifically include methyl, ethyl, propyl and isopropyl, especially preferably methyl.

“Lower alkyl” represented by R² may be substituted with 1 to 3 same or different halogen atoms.

Examples of the substituents include fluorine, chlorine and bromine atoms.

Lower alkyl groups substituted with 1 to 3 same or different halogen atoms include, e.g., fluoromethyl, chloromethyl, bromomethyl and trifluoromethyl.

R² is preferably methyl or fluoromethyl, more preferably methyl.

R³ and R⁴ each independently represent lower alkyl.

Lower alkyl groups represented by R³ and R⁴ include the same groups as the lower alkyl defined above.

Specific examples of the lower alkyl include methyl, ethyl, propyl and isopropyl. The lower alkyl group may be same or different.

It is preferred that both R³ and R⁴ be methyl.

R⁵ is a group selected from the group consisting of:

(1) phenyl, which may be substituted with 1 to 3 same or different groups selected from the group consisting of halogen atoms, lower alkoxy and trifluoromethyl;
(2) heteroaryl selected from the group consisting of pyridinyl, pyrazinyl, imidazolyl, thiazolyl, thienyl and oxazolyl, which heteroaryl may be substituted with 1 to 3 same or different halogen atoms or lower alkyl groups which may be substituted with 1 to 3 same or different halogen atoms;
(3) —O—C₃₋₆ branched lower alkyl, which may be substituted with 1 to 3 same or different halogen atoms; and
(4) C₃₋₇ cycloalkyl, which may be substituted with trifluoromethyl.

Phenyl represented by R⁵ may be substituted with 1 to 3 same or different groups selected from the group consisting of halogen atoms, lower alkoxy and trifluoromethyl.

Halogen atoms of the substituents include the same groups as the halogen atoms defined above.

Lower alkoxy groups of the substituents include the same groups as the lower alkoxy defined above, specifically, e.g., methoxy, ethoxy, propoxy and isopropoxy.

Heteroaryl selected from the group consisting of pyridinyl, pyrazinyl, imidazolyl, thiazolyl, thienyl and oxazolyl, represented by R⁵, may be substituted with 1 to 3 same or different halogen atoms or lower alkyl groups.

Halogen atoms of the substituents include the same groups as the halogen atoms defined above, specifically, e.g., fluorine, chlorine, bromine and iodine atoms.

Lower alkyl groups of the substituents include the same groups as the lower alkyl defined above, specifically, e.g., methyl, ethyl, propyl, isopropyl and butyl.

The lower alkyl of the substituents may be also substituted with 1 to 3 same or different halogen atoms.

Specific examples of the lower alkyl substituted with 1 to 3 same or different halogen atoms include trifluoromethyl.

Among heteroaryl groups selected from the group consisting of pyridinyl, pyrazinyl, imidazolyl, thiazolyl, thienyl and oxazolyl represented by R⁵, which heteroaryl may be substituted with 1 to 3 same or different halogen atoms or lower alkyl groups which may be substituted with 1 to 3 same or different halogen atoms, the heteroaryl groups selected from the group consisting of the pyridinyl and the imidazolyl, which heteroaryl may be substituted with 1 or 2 same or different groups selected from the group consisting of fluorine, chlorine and methyl groups, are preferred, among which 5-fluoro-2-pyridinyl and 1-methyl-2-imidazolyl are more preferred.

The term “—O—C₃₋₆ branched lower alkyl” represented by R⁵ refers to a group having the C₃₋₆ branched-chain lower alkyl defined above bound to oxygen and specifically encompass, e.g., isopropoxy, sec-butoxy, tert-butoxy and 1-ethylpropoxy.

The —O—C₃₋₆ branched lower alkyl may be substituted with 1 to 3 same or different halogen atoms.

Halogen atoms of the substituents include the same groups as the halogen atoms defined above, specifically, e.g., fluorine, chlorine, bromine and iodine atoms.

As “—O—C₃₋₆ branched lower alkyl, which may be substituted with 1 to 3 same or different halogen atoms” represented by R⁵, tert-butoxy or 1-ethylpropoxy is preferred.

C₃₋₇ cycloalkyl groups represented by R⁵ include the C₃₋₇ cycloalkyl defined above, specifically, e.g., cyclopropyl, cyclobutyl, cyclopentyl, cyclohexyl and, cycloheptyl.

The C₃₋₇ cycloalkyl may be substituted with trifluoromethyl.

As “C₃₋₇ cycloalkyl which may be substituted with trifluoromethyl” represented by R⁵, 1-(trifluoromethyl)cyclopropyl or 4-trifluoromethylcyclohexyl is preferred.

R⁵ is preferably a group selected from the group consisting of phenyl, 4-chlorophenyl, 4-fluorophenyl, 3,4-difluorophenyl, 4-difluoromethoxyphenyl, 4-trifluoromethoxyphenyl, 5-fluoro-2-pyridinyl, 2-fluoro-4-pyridinyl, tert-butoxy, 1-(trifluoromethyl)cyclopropyl and 4-trifluoromethylcyclohexyl, more preferably a group selected from the group consisting of phenyl, 4-fluorophenyl, 4-difluoromethoxyphenyl, 4-trifluoromethoxyphenyl, 5-fluoro-2-pyridinyl, 1-(trifluoromethyl)cyclopropyl and tert-butoxy.

m is an integer of from 0 to 2, where it is preferred that m be 1.

Groups represented by a formula (II):

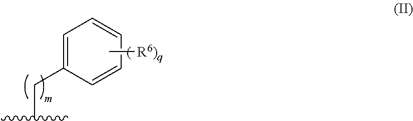

wherein [symbol]
represents a binding site, in the formula (I) are preferably groups selected from the group consisting of 4-trifluoromethoxyphenyl, 3,5-dichlorophenyl, 2-chlorophenyl, 4-fluoro-3-trifluoromethylphenyl, 2-trifluoromethylphenyl, 2-fluorophenyl, 2-trifluoromethoxyphenyl, 4-trifluoromethoxyphenyl, 3,5-difluorophenyl, 3,4-dichlorophenyl, 3,5-dichlorophenyl, 4-chlorophenyl, 4-chloro-3-fluorophenyl, 3,4-difluorophenyl, 3-methylphenyl, 3-trifluoromethoxyphenyl, 3,5-bis(trifluoromethyl)phenyl and 3-chloro-5-fluorophenyl.

Specifically, examples of compounds according to the present invention include, but are not limited to,

4-fluoro-N-[2-({7-fluoro-1-methyl-2-oxo-5-[4-(trifluoromethoxy)benzyl]-2,3-dihydro-1H-1,4-benzo diazepin-3-yl}amino)-1,1-dimethyl-2-oxoethyl]benzamide,
N-[2-(7-fluoro-1-methyl-2-oxo-5-[4-(trifluoromethoxy)benzyl]-2,3-dihydro-1H-1,4-benzodiazepin-3-yl}amino)-1,1-dimethyl-2-oxoethyl]benzamide,
N-[1,1-dimethyl-2-({1-methyl-2-oxo-5-[4-(trifluoromethoxy)benzyl]-2,3-dihydro-1H-1,4-benzodiaz epin-3-yl}amino)-2-oxoethyl]-4-fluorobenzamide and
N-[1,1-dimethyl-2-({1-methyl-2-oxo-5-[4-(trifluoromethoxy)benzyl]-2,3-dihydro-1H-1,4-benzodiaz epin-3-yl}amino)-2-oxoethyl]benzamide.

In accordance with a preferred embodiment, any aspects of R¹, R², R³, R⁴, R⁵, R⁶, p, q and m as described above may be combined.

A process for producing a compound according to the present invention will now be described.

A compound represented by the formula (I):

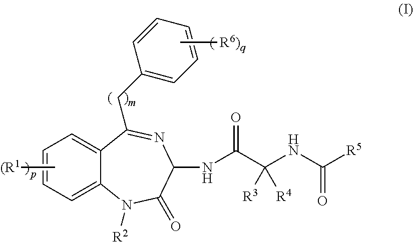

wherein each symbol has the same definition specified above, in accordance with the present invention, can be produced, e.g., by the following process:

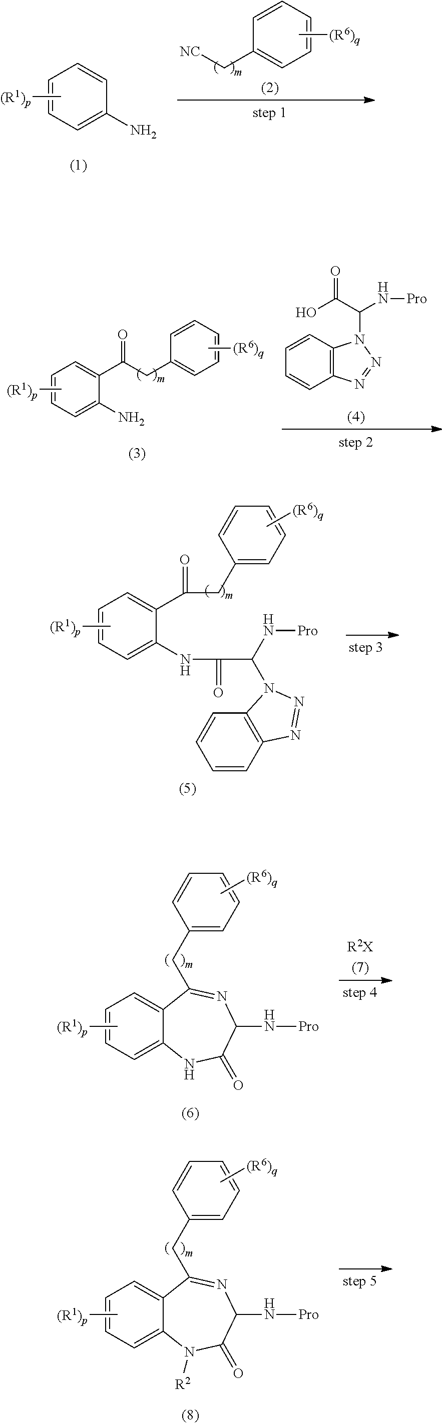

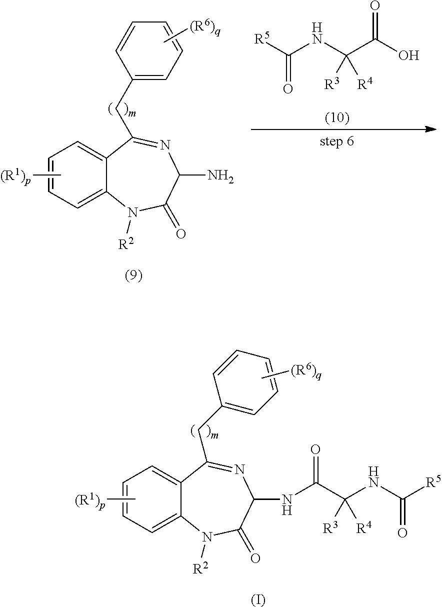

wherein X₁ is a leaving group; Pro is a protective group of amino; and the other symbols have the same definitions specified above, processes described in the Examples or other process known in the art.

### Step 1

This step is a process for producing a compound (3) by reacting a compound (1) with a compound (2) in the presence of Lewis acid.

Examples of Lewis acids as used in this step include aluminum chloride, zinc chloride, boron trichloride and mixtures thereof.

An amount of Lewis acid used is typically 1-5 equivalents, preferably 1-2 equivalents, per equivalent of the compound (1).

An amount of the compound (2) used is typically 1-3 equivalents, preferably 1-1.5 equivalents, per equivalent of the compound (1).

Unless interfering with the reaction, any solvent may be used in this step, examples of which include dichloromethane and dichloroethane.

The reaction temperature is typically 0-150° C., preferably 40-80° C.

The reaction time is typically 1-48 hours, preferably 1-12 hours.

The compound (3) obtained in such a manner may be isolated and purified by well-known separation and purification measures such as concentration, vacuum concentration, reprecipitation, solvent extraction, crystallization and chromatography, or the isolation and purification may be omitted to subject the compound (3) to the subsequent step.

### Step 2

This step is a process for producing a compound (5) by reacting the compound (3) with an amino-protected α-(1-benzotriazolyl)glycine derivative (4).

For this reaction, typical amide formation reaction may be performed by methods as described in documents (e.g., Nobuo Izumiya, et al.: Peptide Gosei no Kiso to Jikken (Fundamentals and Experiments of Peptide Synthesis), Maruzen (1983); Comprehensive Organic Synthesis, Vol. 6, Pergamon Press (1991), etc.), other methods known in the art or combinations thereof, that is, by using a condensation agent that is well known to those skilled in the art, or by an ester activation method, a mixed anhydride method, an acid chloride method, a carbodiimide method, etc., which can be used by those skilled in the art. Examples of such amide formation reagents include thionyl chloride, oxalyl chloride, N,N-dicyclohexylcarbodiimide, 1-methyl-2-bromopyridinium iodide, N,N′-carbonyldiimidazole, diphenylphosphoryl chloride, diphenylphosphoryl azide, N,N′-disuccinimidyl carbonate, N,N′-disuccinimidyl oxalate, 1-ethyl-3-(3-dimethylaminopropyl)carbodiimide hydrochloride, ethyl chloroformate, isobutyl chloroformate and benzotriazol-1-yl-oxy-tris(dimethylamino)phosphonium hexafluorophosphate; especially preferably, e.g., oxalyl chloride, 1-ethyl-3-(3-dimethylaminopropyl)carbodiimide hydrochloride, N,N-dicyclohexylcarbodiimide and benzotriazol-1-yl-oxy-tris(dimethylamino)phosphonium hexafluorophosphate.

For the amide formation reaction, base and a condensation adjuvant may be also used together with the amide formation reagent.

Bases as used include ternary aliphatic amines such as trimethylamine, triethylamine, N,N-diisopropylethylamine, N-methylmorpholine, N-methylpyrrolidine, N-methylpiperidine, N,N-dimethylaniline, 1,8-diazabicyclo[5.4.0]undeca-7-en (DBU) and 1,5-azabicyclo[4.3.0]nona-5-en (DBN); and aromatic amines such as pyridine, 4-dimethylaminopyridine, picoline, lutidine, quinoline and isoquinoline; especially preferably, N-methylmorpholine.

Condensation adjuvants as used include, for example, N-hydroxybenzotriazole hydrate, N-hydroxy succinimide, N-hydroxy-5-norbornen-2,3-dicarboximide and 3-hydroxy-3,4-dihydro-4-oxo-1,2,3-benzotriazole; especially preferably, e.g., N-hydroxybenzotriazole, etc.

Protective groups Pro for the amino group of the compound (4) include groups as described in documents (e.g., T. W. Green: Protective Groups in Organic Synthesis; Second Edition, John Wiley & Sons (1991), etc.), specifically, e.g., benzyloxycarbonyl.

An amount of the compound (3) used is typically 1-10 equivalents, preferably 1-3 equivalents, per equivalent of the compound (4) or a reactive derivative thereof.

An amount of the base used is typically 1-10 equivalents, preferably 1-5 equivalents, depending on the types of a compound and a solvent used and other reaction conditions, per equivalent of the compound (4).

Unless interfering with the reaction, reaction solvents that can be used in this step, include, but are not limited to, e.g., inactive solvents; specifically, e.g., chloroform, 1,2-dichloroethane, xylene, toluene, 1,4-dioxane, tetrahydrofuran and dimethoxyethane or mixed solvents thereof, among which tetrahydrofuran is preferred.

The reaction time is typically 0.5-96 hours, preferably 3-24 hours.

The reaction temperature is typically from 0° C. to the boiling point of a solvent, preferably from room temperature to 80° C.

One or a combination of two or more of bases, amide formation reagents and condensation adjuvants as used in this step may be used.

The compound (5) obtained in such a manner may be isolated and purified by well-known separation and purification measures such as concentration, vacuum concentration, crystallization, solvent extraction, reprecipitation and chromatography, or the isolation and purification may be omitted to subject the compound (5) to the subsequent step.

### Step 3

This step is a process of obtaining an aminoketone derivative by reacting the compound (5) with ammonia and thereafter cyclizing the aminoketone derivative with an acid catalyst to produce a compound (6).

An amount of ammonia used is typically 1-100 equivalents, preferably 10-50 equivalents per equivalent of the compound (5).

Unless interfering with the reaction, any reaction solvent may be used, examples of which include methanol and ethanol.

The reaction temperature is typically 0-100° C., preferably 20-50° C.

The reaction time is typically 1-24 hours, preferably 1-12 hours.

A compound (A) obtained in such a manner

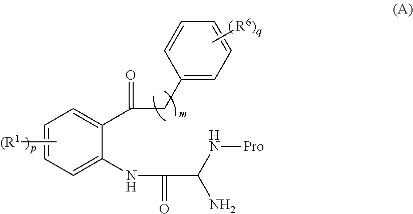

may be isolated and purified by well-known separation and purification measures such as concentration, vacuum concentration, reprecipitation, solvent extraction, crystallization and chromatography, or the isolation and purification may be omitted to subject the compound (A) to the subsequent step.

In the process, the compound (A) is then cyclized in the presence of an acid catalyst to produce the compound (6).

Acid catalysts as used in this step specifically include, e.g., acetic acid and ammonium acetate.

An amount of the acid catalyst used is typically 0.1-10 equivalents, preferably 0.1-1 equivalent per equivalent of the compound (A).

Unless interfering with the reaction, any reaction solvent may be used, examples of which include water, methanol, ethanol, methylene chloride, dimethylformamide and acetic acid.

The reaction time is typically 0.5-24 hours, preferably 0.5-12 hours.

The reaction temperature is typically 0-100° C., preferably 0-50° C.

The compound (6) obtained in such a manner may be isolated and purified by well-known separation and purification measures such as concentration, vacuum concentration, reprecipitation, solvent extraction, crystallization and chromatography, or the isolation and purification may be omitted to subject the compound (6) to the subsequent step.

### Step 4

This step is a process of producing a compound (8) by reacting the compound (6) with the compound (7) in the presence of base.

Bases as used include, for example, sodium-tert-pentoxide, sodium-tert-butoxide, potassium-tert-butoxide and sodium hydride.

An amount of the base used is typically 1-3 equivalents, preferably 1-1.5 equivalents, per equivalent of the compound (6).

X in the compound (7) represents a leaving group, of which examples include bromine and iodine atoms.

An amount of the compound (7) used is typically 1-3 equivalents, preferably 1-1.5 equivalents, per equivalent of the compound (6).

Unless interfering with the reaction, any reaction solvent may be used, examples of which include dimethylformamide and tetrahydrofuran.

The reaction time is typically 1-24 hours, preferably 1-12 hours.

The reaction temperature is typically from 0-100° C., preferably 0-50° C.

The compound (8) obtained in such a manner may be isolated and purified by well-known separation and purification measures such as concentration, vacuum concentration, reprecipitation, solvent extraction, crystallization and chromatography, or the isolation and purification may be omitted to subject the compound (8) to the subsequent step.

### Step 5

This step is a process of producing a compound (9) by removing a protective group Pro for the amino group of the compound (8).

The reaction in this step can be carried out by methods as described in documents (e.g., T. W. Green: Protective Groups in Organic Synthesis, Second Edition, John Wiley & Sons (1991), etc.), methods equivalent thereto or combinations of these with usual methods. When benzyloxycarbonyl is used as Pro, the protective group may be removed with, e.g., hydrogen bromide-acetic acid solution.

The compound (9) obtained in such a manner may be isolated and purified by well-known separation and purification measures such as concentration, vacuum concentration, reprecipitation, solvent extraction, crystallization and chromatography, or the isolation and purification may be omitted to subject the compound (9) to the subsequent step.

### Step 6

This step is a process for producing a compound (1) by reacting the compound (9) with a compound (10).

For this reaction, typical amide formation reaction may be performed by methods as described in documents (e.g., Nobuo Izumiya, et al.: Peptide Gosei no Kiso to Jikken (Fundamentals and Experiments of Peptide Synthesis), Maruzen (1983); Comprehensive Organic Synthesis, Vol. 6, Pergamon Press (1991), etc.), other methods known in the art or combinations thereof, that is, by using a condensation agent that is well known to those skilled in the art, or by an ester activation method, a mixed anhydride method, an acid chloride method, a carbodiimide method, etc., which can be used by those skilled in the art. Examples of such amide formation reagents include thionyl chloride, oxalyl chloride, N,N-dicyclohexylcarbodiimide, 1-methyl-2-bromopyridinium iodide, N,N′-carbonyldiimidazole, diphenylphosphoryl chloride, diphenylphosphoryl azide, N,N′-disuccinimidyl carbonate, N,N′-disuccinimidyl oxalate, 1-ethyl-3-(3-dimethylaminopropyl)carbodiimide hydrochloride, ethyl chloroformate, isobutyl chloroformate and benzotriazol-1-yl-oxy-tris(dimethylamino)phosphonium hexafluorophosphate; especially preferably, e.g., thionyl chloride, 1-ethyl-3-(3-dimethylaminopropyl)carbodiimide hydrochloride, N,N-dicyclohexylcarbodiimide and benzotriazol-1-yl-oxy-tris(dimethylamino)phosphonium hexafluorophosphate. For the amide formation reaction, base and a condensation adjuvant may be also used together with the amide formation reagent.

Bases as used include ternary aliphatic amines such as trimethylamine, triethylamine, N,N-diisopropylethylamine, N-methylmorpholine, N-methylpyrrolidine, N-methylpiperidine, N,N-dimethylaniline, 1,8-diazabicyclo[5.4.0]undeca-7-en (DBU) and 1,5-azabicyclo[4.3.0]nona-5-en (DBN); and aromatic amines such as pyridine, 4-dimethylaminopyridine, picoline, lutidine, quinoline and isoquinoline; especially preferably, e.g., ternary aliphatic amines, etc., particularly preferably, e.g., trimethylamine, N,N-diisopropylethylamine, etc.

Condensation adjuvants as used include, for example, N-hydroxybenzotriazole hydrate, N-hydroxy succinimide, N-hydroxy-5-norbornen-2,3-dicarboximide and 3-hydroxy-3,4-dihydro-4-oxo-1,2,3-benzotriazole; especially preferably, e.g., N-hydroxybenzotriazole, etc.

An amount of the compound (9) used is typically 1-10 equivalents, preferably 1-3 equivalents, per equivalent of the compound (10) or a reactive derivative thereof.

An amount of the base used is typically 1-10 equivalents, preferably 1-5 equivalents per equivalent of the compound (10) or a reactive derivative thereof.

Unless interfering with the reaction, reaction solvents that can be used in this step, include, but are not limited to, e.g., inactive solvents; specifically, e.g., methylene chloride, chloroform, 1,2-dichloroethane, dimethylformamide, ethyl acetate, methyl acetate, acetonitrile, benzene, xylene, toluene, 1,4-dioxane, tetrahydrofuran and dimethoxyethane or mixed solvents thereof; preferably, e.g., methylene chloride, chloroform, 1,2-dichloroethane, acetonitrile and N,N-dimethylformamide, from the viewpoint of ensuring preferable reaction temperature.

The reaction time is typically 0.5-96 hours, preferably 3-24 hours.

The reaction temperature is typically from 0° C. to the boiling point of a solvent, preferably from room temperature to 80° C.

One or a combination of two or more of bases, amide formation reagents and condensation adjuvants as used in this step may be used.

The compound (1) obtained in such a manner may be isolated and purified by well-known separation and purification measures such as concentration, vacuum concentration, crystallization, solvent extraction, reprecipitation and chromatography.

The 1,4-benzodiazepine-2-on derivative in accordance with the present invention may be present as a pharmaceutically acceptable salt, which may be produced according to usual methods using the compound (1).

Examples of such acid addition salts include hydrohalic acid salts such as hydrochloride, hydrofluorate, hydrobromide and hydroiodide; inorganic acid salts such as nitride, perchlorate, sulfate, phosphate and carbonate; lower alkyl sulfonate salts such as methanesulfonate, trifluoromethanesulfonate and ethanesulfonate; aryl sulfonates such as benzenesulfonate and p-toluenesulfonate; organic salts such as fumarate, succinate, citrate, tartrate, oxalate and maleate; and acid addition salts of organic acids, e.g., amino acids, such as glutamate and aspartate.

When the compound according to the present invention has an acidic group, such as carboxyl, in the group, the compound can be also converted into a corresponding pharmaceutically acceptable salt by processing the compound with base. Examples of such base addition salts include alkali metal salts such as sodium and potassium; alkaline earth metal salts such as calcium and magnesium; ammonium salts; and salts of organic bases such as guanidine, triethylamine and dicyclohexylamine.

Furthermore, the compound according to the present invention may be present in the form of a free compound or any hydrate or solvate of a salt thereof.

In contrast, a salt or ester can be also converted into a free compound by a usual method.

Furthermore, in the compound according to the present invention, a stereoisomer or a tautomer, such as an optical isomer, a diastereoisomer or a geometrical isomer, is sometimes present depending on the form of a substituent. It will be appreciated that these isomers are encompassed entirely by compounds according to the present invention. Furthermore, it will be appreciated that any mixture of these isomers is encompassed by compounds according to the present invention.

A compound represented by the general formula (I) may be orally or parenterally administered and is formulated into a form suitable for such administration to provide an agent for treating and/or preventing hyperlipidemia, diabetes and obesity using the compound.

When the compound according to the present invention is clinically used, a pharmaceutically acceptable additive may be also added, depending on a dosage form, to produce various preparations, followed by administration of the preparations. Additives in this case, for which various additives that are usually used in the field of formulation, include, for example, gelatine, lactose, saccharose, titanium oxide, starch, microcrystalline cellulose, hydroxypropyl methylcellulose, carboxymethyl cellulose, corn starch, microcrystalline wax, white petrolatum, magnesium aluminometasilicate, anhydrous calcium phosphate, citric acid, trisodium citrate, hydroxypropylcellulose, sorbitol, sorbitan fatty acid esters, polysorbates, sucrose fatty acid esters, polyoxyethylene, hydrogenated castor oil, polyvinyl pyrrolidone, magnesium stearate, light anhydrous silicic acid, talc, vegetable oil, benzyl alcohol, gum arabic, propylene glycol, polyalkylene glycol, cyclodextrin, hydroxypropyl cyclodextrin, etc.

Examples of dosage forms as formulated mixtures with such additives include solid preparations such as tablets, capsules, granules, powders and suppositories; and liquid preparations such as syrups, elixirs and injections, which can be prepared according to typical methods in the field of formulation. Further, the liquid preparations may be in the form of dissolution or suspension in water or another appropriate medium just before use. Particularly, the injections may be also dissolved or suspended in a physiological saline solution or a glucose solution as needed, and a buffer or a preservative may be further added to the mixture.

Such preparations may contain the compound according to the present invention at a rate of 1.0-100%, preferably 1.0-60%, by weight of the total drug. Such preparations may also contain other therapeutically-effective compounds.

The compound according to the present invention may be used in combination with a drug efficacious for hyperlipidemia, diabetes, obesity or the like (hereinafter referred to as “concomitant drug”). Such drugs may be administered concurrently, separately or sequentially in treatment or prevention of the diseases. When the compound according to the present invention is used concurrently with one or more concomitant drugs, they may be formed into a pharmaceutical composition in a single dosage form. In a combination therapy, however, a composition containing the compound according to the present invention and a concomitant drug in different packages may be administered concurrently, separately or sequentially to an administration subject. They may be also administered at intervals.

A dose of a concomitant drug may be based on a dose which is clinically used and may be selected appropriately depending on an administration subject, an administration route, a disease, a combination and the like. A dosage form of such a concomitant drug is not particularly limited, and it may be any form in which the compound according to the present invention and a concomitant drug are combined when they are administered. Examples of such dosage forms include (1) administration of a single pharmaceutical preparation obtained by formulating the compound according to the present invention and a concomitant drug concurrently; (2) coadministration via the same administration route of two pharmaceutical preparations obtained by formulating the compound according to the present invention and a concomitant drug separately; (3) administration at an interval via the same administration route of two pharmaceutical preparations obtained by formulating the compound according to the present invention and a concomitant drug separately; (4) coadministration via different administration routes of two pharmaceutical preparations obtained by formulating the compound according to the present invention and a concomitant drug separately; and (5) administration at an interval via different administration routes of two pharmaceutical preparations obtained by formulating the compound according to the present invention and a concomitant drug separately (e.g. administration of the compound according to the present invention and then a concomitant drug, or administration in the reverse order). The blending ratio of the compound according to the present invention and a concomitant drug may be selected appropriately depending on an administration subject, an administration route, a disease, and the like.

When the compound according to the present invention is used in clinical fields, a dosage regimen of it depends on the sex, age, body weight and severity of condition of a patient; and the type and range of desired therapeutic effect. In case of oral administration to an adult human, the usual dosage regimen of it is 0.01-100 mg/kg per day, preferably 0.03-1 mg/kg per day in one dose or several divided doses. In case of parenteral administration, it is 0.001-10 mg/kg per day, preferably 0.001-0.1 mg/kg per day in one dose or several divided doses.

Any appropriate administration route may be used to administer an effective amount of the compound according to the present invention to a mammal, particularly to a human. For example, oral, rectum, local, intravenous, ocular, lung and nasal administration routes may be used. Examples of dosage forms include tablets, troches, powders, suspensions, solutions, capsules, creams, aerosols, etc., in which tablets for oral use are preferred.

For preparing compositions for oral use, any typical pharmaceutical medium may be used, examples of which include water, glycol, oils, alcohols, flavoring agents, preservatives, coloring agents, etc. For preparing liquid compositions for oral use, examples of pharmaceutical media include suspensions, elixirs and solutions, and examples of carriers include starches, sugars, microcrystalline celluloses, diluents, granulating agents, lubricants, binders and disintegrating agents. For preparing solid compositions for oral use, examples of pharmaceutical media include powders, capsules and tablets. Particularly, the solid compositions for oral use are preferred.

Because of their ease of administration, tablets and capsules represent the most advantageous oral dosage unit form. If desired, tablets can be coated with standard aqueous or non-aqueous techniques.

In addition to the common dosage forms described above, the compounds according to the formula (I) may also be administered by controlled release means and/or delivery devices that are described in U.S. Pat. Nos. 3,845,770; 3,916,899; 3,536,809; 3,598,123; 3,630,200 and 4,008,719.

Pharmaceutical compositions in accordance with the present invention suitable for oral administration include capsules, cachets or tablets, each containing a predetermined amount of an active ingredient, such as a powder or granules, or as an aqueous liquid, a non-aqueous liquid, an oil-in-water emulsion or a water-in-oil liquid emulsion. Such compositions may be prepared by any pharmaceutical method, including a method of combining an active ingredient with a carrier consisting of one or more necessary constituents.

In general, compositions are prepared by uniformly and sufficiently mixing active ingredients with liquid carriers or finely divided solid carriers, or both, and then shaping the product into the desired form if necessary. For example, a tablet can be prepared optionally together with one or more accessory ingredients by compression or molding. Compressed tablets can be prepared by compressing, in a suitable machine, the active ingredients in a free-flowing form such as powder or granules, optionally mixed with a binder, a lubricant, an inert excipient, a surfactant or a dispersive agent.

Molded tablets can be prepared by molding, in a suitable machine, a mixture of the powdered compound moistened with an inert liquid diluent.

Preferably, each tablet contains about 1 mg to 1 g of active ingredient, and each cachet or capsule contains about 1 mg to 500 mg of active ingredient.

Examples of pharmaceutical dosage forms for the compound of the formula (I) are shown below.

TABLE1

| Suspension for injection | |
|---|---|
| (I. M.) | mg/ml |
| Compound of formula (I) | 10 |
| Methyl cellulose | 5.0 |
| Tween 80 | 0.5 |
| Benzyl alcohol | 9.0 |
| Benzalkonium chloride | 1.0 |

Adjusted to 1.0 ml by addition of water for
injection.

TABLE 2

| Tablet | mg/tablet | |
|---|---|---|
| Compound of formula (I) | 25 | |
| Methyl cellulose | 415 | |
| Tween 80 | 14.0 | |
| Benzyl alcohol | 43.5 | |
| Magnesium stearate | 2.5 | |
| Total | 500 | mg |

TABLE 3

| Capsule | mg/capsule | |
|---|---|---|
| Compound of formula (I) | 25 | |
| Lactose powder | 573.5 | |
| Magnesium stearate | 1.5 | |
| Total | 600 | mg |

TABLE 4

| Aerosol | Per container | |
|---|---|---|
| Compound of formula (I) | 24 | mg |
| Lecithin, NF Liq. Conc | 1.2 | mg |
| Trichlorofluoromethane,NF | 4.025 | g |
| Dichlorodifluoromethane,NF | 12.15 | g |

The compound of the formula (I) may be used in combination with other drugs used in treatment/prevention/delay of onset of hyperlipidemia, diabetes or obesity as well as diseases or conditions associated therewith. The other drugs may be administered in an administration route or a dose that is typically used, concurrently with or separately from the compound of the formula (I).

When the compound of the formula (I) is used concurrently with one or more drugs, a pharmaceutical composition containing the compound of the formula (I) and the other drugs is preferred.

Accordingly, the pharmaceutical composition according to the present invention contains the compound of the formula (I) as well as other active ingredients that are one or more. Examples of active ingredients which are used in combination with the compound of the formula (I) include, but are not limited to, the following (a) to (j):

(a) other DGAT1 inhibitors;
(b) glucokinase activators;
(c) biguanides (e.g., buformin, metformin and phenformin);
(d) PPAR agonists (e.g., troglitazone, pioglitazone and rosiglitazone);
(e) insulin;
(f) somatostatin;
(g) α-glucosidase inhibitors (e.g., voglibose, miglitol and acarbose);
(h) insulin secretagogues (e.g., acetohexamide, carbutamide, chlorpropamide, glybenclamide, gliclazide, glimepiride, glipizide, gliquidone, glisoxepide, glyburide, glyhexamide, glypinamide, phenbutamide, tolazamide, tolbutamide, tolcyclamide, nateglinide and repaglinide);
(i) DPP-IV (dipeptidyl peptidase-IV) inhibitors; and
(j) glucose uptake facilitators,
which may be administered separately or in the same pharmaceutical composition.

A weight ratio of the compound of the formula (I) to a second active ingredient varies within wide limits and further depends on the effective dose of each active ingredient. Accordingly, for example, when the compound of the formula (I) is used in combination with a PPAR agonist, a weight ratio of the compound of the formula (I) to the PPAR agonist is generally about 1000:1 to 1:1000, preferably about 200:1 to 1:200. Combinations of the compound of the formula (I) and other active ingredients are within the above-mentioned range; and in any case, the effective dose of each active ingredient should be used.

The present invention is described below in more detail referring to Examples and Reference Examples, but is not limited thereto.

The compound according to the present invention or a pharmaceutically acceptable salt thereof has strong DGAT1 inhibitory activity and is thus useful for treating and/or preventing hyperlipidemia, diabetes and obesity.

It should be understood by those skilled in the art that various modifications, combinations, sub-combinations and alterations may occur depending on design requirements and other factors insofar as they are within the scope of the appended claims or the equivalents thereof.

### EXAMPLES

Wakogel (registered trademark) C-300, made by Wako Pure Chemical Industries Ltd., or KP-Sil (Registered Trademark) Silica prepacked column, made by Biotage, was used for the silica gel column chromatography in Examples. Kieselgel™60 F₂₅₄, Art. 5744, made by Merck & Co., was used for preparative thin layer chromatography. Chromatorex (registered trademark) NH (100-250 mesh or 200-350 mesh), made by Fuji Silysia Chemical Ltd., was used for basic silica gel column chromatography.

¹H-NMR was measured using Gemini (200 MHz, 300 MHz), Mercury (400 MHz) and Inova (400 MHz), made by Varian, using tetramethylsilane as a standard substance. In addition, the mass spectra were measured by electrospray ionization (ESI) or atmospheric pressure chemical ionization (APCI) using Micromass ZQ made by Waters.

The meanings of the abbreviations in Examples are shown below.

i-Bu=isobutyl
n-Bu=n-butyl
t-Bu=tert-butyl
Boc=tert-butoxycarbonyl
Me=methyl
Et=ethyl
Ph=phenyl
i-Pr=isopropyl
n-Pr=n-propyl
CDCl₃=heavy chloroform
CD₃OD=heavy methanol
DMSO-d₆=heavy dimethylsulfoxide

The meanings of the abbreviations in the nuclear magnetic resonance spectra are shown below.

s=singlet
d=doublet
dd=double doublet
dt=double triplet
ddd=double double doublet
Sept=septet
t=triplet
m=multiplet
br=broad
brs=broad singlet
q=quartet
J=coupling constant
Hz=hertz

### Example 1

### 4-fluoro-N-[2-({7-fluoro-1-methyl-2-oxo-5-[4-(trifluoromethoxy)benzyl]-2,3-dihydro-1H-1,4-benzo diazepin-3-yl}amino)-1,1-dimethyl-2-oxoethyl]benzamide (enantiomers A and B)

### Step 1

### Synthesis of 1-(2-amino-5-fluorophenyl)-2-[4-(trifluoromethoxy)phenyl]ethanone

A solution of 1M chloride boron in dichloromethane (66 ml, 66 mmol) was diluted with 250 ml of 1,2-dichloroethane, and 4-fluoroaniline (6.67 g, 60 mmol) was added under ice-cooling. The mixture was stirred for 30 minutes, followed by sequentially adding 4-trifluoromethoxybenzyl nitrile (30 g 150 mmol) and aluminum chloride (8.8 g, 66 mmol) and thereafter heating the mixture under reflux with stirring for 14 hours. An aqueous 2N-hydrochloric acid solution (60 ml, 120 mmol) was added at room temperature, followed by stirring the mixture at 80° C. for 30 minutes. The reaction solution was extracted with chloroform, dried over sodium sulfate, filtered, and thereafter concentrated. The resultant crude product was purified by silica gel column chromatography and thereafter crystallized to yield the title compound (2.44 g, 7.8 mmol) as a yellow crystal.

### Step 2

### Synthesis of

### benzyl {7-fluoro-2-oxo-5-[4-(trifluoromethoxy)benzyl]-2,3-dihydro-1H-1,4-benzodiazepin-3-yl}carbamate

In 60 ml of THF, 2-(benzotriazol-1-yl)-N-(benzylcarbonyl)glycine (5.73 g, 17 mmol) was dissolved, oxalyl chloride (2.3 g, 18 mmol) was dropped under ice-cooling, DMF (0.11 g, 1.6 mmol) was subsequently added, the mixture was stirred at unprocessed temperature for 1 hour, thereafter oxalyl chloride (0.23 g, 1.8 mmol) was readded, and the mixture was further stirred for 2 hours. Subsequently, 1-(2-amino-5-fluorophenyl)-2-[4-(trifluoromethoxy)phenyl]ethanone (5.0 g) and N-methylmorpholine (4.2 ml, 38 mmol) were dissolved in 5 ml of THF, and the mixture was dropped under ice-cooling. A reaction mixture was stirred under room temperature for 2 hours, followed by removing the resulting precipitates by filtration. Ammonia gas was blown into the resultant filtrate for 10 minutes, thereafter 35 ml of methanol was added, ammonia gas was reblown for 30 minutes, and the mixture was stirred overnight at room temperature. The reaction mixture was concentrated and dissolved in 40 ml of acetic acid, the mixture was stirred for 3 hours, thereafter ammonium acetate (5.8 g, 75 mmol) was added, and the mixture was further stirred for 1 hour. The reaction liquid was diluted with ethyl acetate and was basified using a saturated aqueous sodium bicarbonate solution. After extraction, the organic layer was washed with a saturated saline solution, dried over sodium sulfate, filtered, concentrated, purified using silica gel chromatography, and thereafter crystallized with ethyl acetate and hexane to yield the title compound (4.8 g, 9.57 mmol) as a colorless solid.

### Step 3

### Synthesis of

### benzyl {7-fluoro-1-methyl-2-oxo-5-[4-trifluoromethoxy)benzyl]-2,3-dihydro-1H-1,4-benzodiazepin-3-yl}carbamate

Benzyl {7-fluoro-2-oxo-5-[4-(trifluoromethoxy)benzyl]-2,3-dihydro-1H-1,4-benzodiazepin-3-yl}carbamate (5.0 g, 10 mmol) was dissolved in DMF 50 ml, sodium tert-pentoxide (1.15 g, 10.5 mmol) and methyl iodide (1.49 g, 10.5 mmol) were sequentially added under ice-cooling, the mixture was stirred under ice-cooling for 1 hour, thereafter sodium tert-pentoxide (0.11 g, 1.0 mmol) and methyl iodide (0.14 g, 1.0 mmol) were sequentially added again, and the mixture was further stirred under ice-cooling for 30 minutes.

The mixture was quenched using a saturated aqueous ammonium chloride solution, diluted with ethyl acetate, and thereafter washed with water and saturated saline solution. The washed matter was dried over sodium sulfate, thereafter filtered, concentrated, and purified by silica gel chromatography to yield the title compound (5.06 g, 9.8 mmol) as a colorless amorphous.

### Step 4

### Synthesis of

### 3-amino-7-fluoro-1-methyl-5-[4-(trifluoromethoxy)benzyl]-1,3-dihydro-2H-1,4-benzodiazepin-2-on

Benzyl {7-fluoro-1-methyl-2-oxo-5-[4-(trifluoromethoxy)benzyl]-2,3-dihydro-1H-1,4-benz odiazepin-3-yl}carbamate (4.46 g) was dissolved in 30% hydrogen bromide-acetic acid solution and the mixture was stirred at room temperature for 30 minutes. The mixture was diluted with water, washed with diethyl ether, and the aqueous layer was then basified using a saturated aqueous sodium bicarbonate solution and extracted with ethyl acetate. The organic layer was washed with a saturated aqueous sodium chloride solution, thereafter dried over sodium sulfate, filtered and concentrated to yield the title compound (3.05 g, 8.0 mmol) as a yellow oily substance.

### Step 5

### Synthesis of

### 4-fluoro-N-[2-({7-fluoro-1-methyl-2-oxo-5-[4-(trifluoromethoxy)benzyl]-2,3-dihydro-1H-1,4-benzo diazepin-3-yl}amino)-1,1-dimethyl-2-oxoethyl]benzamide

In DMF 5 ml, 3-amino-7-fluoro-1-methyl-5-[4-(trifluoromethoxy)benzyl-1,3-dihydro-2H-1,4-benzodiazepin-2-on (150 mg, 0.39 mmol), WSC (134 mg, 0.59 mmol), HOBT (80 mg, 0.59 mmol) and 2-{[(4-fluorophenyl)]carbonyl]amino}-2-methyl propionic acid (133 mg, 0.59 mmol) were dissolved, and the mixture was stirred under nitrogen atmosphere for 3 hours. After dilution with ethyl acetate, the mixture was washed with water, an aqueous hydrochloric acid solution, a saturated aqueous sodium bicarbonate solution and a saturated aqueous sodium chloride solution sequentially, and dried over sodium sulfate. After filtration and concentration, the mixture was purified by silica gel chromatography to yield the title compound (170 mg, 0.29 mmol) as a racemate. Optical resolution of the resultant racemate (150 mg) was carried out by chiral column chromatography (Daicel CHIRALPAK IA (20*250 mm, 5 μm), hexane:isopropyl alcohol=70:30) to yield enantiomer A (faster: 56 mg) and enantiomer B (slower: 58 mg) of the title compound as colorless amorphous, respectively.

The analytical data of the title compound are shown below.

¹H-NMR (CDCl3) δ: 1.77 (3H, s), 1.78 (3H, s), 3.20 (3H, s), 4.01 (1H, d, J=14 Hz), 4.06 (1H, d, J=14 Hz), 5.34 (1H, d, J=7 Hz), 7.01-7.09 (7H, m), 7.15-7.24 (3H, m), 7.71-7.79 (3H, m).

ESI-MS (m/e): 589 [M+H]+

### Example 2

### N-[2-({7-fluoro-1-methyl-2-oxo-5-[4-(trifluoromethoxy)benzyl]-2,3-dihydro-1H-1,4-benzodiazepin 3-yl}amino)-1,1-dimethyl-2-oxoethyl]benzamide (enantiomers A and B)

Using 2-methyl-2-[(phenylcarbonyl)amino)propionic acid, the title compound was obtained as a racemate by the same method as in Example 1 (Step 5).

Optical resolution of the resultant racemate was carried out by chiral column chromatography (Daicel CHIRALPAK AD-H (20*250 mm, 5 μm), hexane:isopropyl alcohol=60:40) to yield enantiomer A (faster: 58 mg) and enantiomer B (slower: 60 mg) of the title compound as colorless amorphous, respectively.

The analytical data of the title compound are shown below.

¹H-NMR (CDCl3) δ: 1.77 (3H, s), 1.79 (3H, s), 3.18 (3H, s), 4.01 (1H, d, J=14.0 Hz), 4.06 (1H, d, J=14.0 Hz), 5.3 5 (1H, d, J=7.2 Hz), 7.00-7.04 (3H, m), 7.08 (2H, d, J=9 Hz), 7.16-7.23 (3H, m), 7.36-7.49 (3H, m), 7.73-7.79 (3H, m).

ESI-MS (m/e): 571 [M+H]⁺

### Example 3

### N-[1,1-dimethyl-2-({1-methyl-2-oxo-5-[4-(trifluoromethoxy)benzyl]-2,3-dihydro-1H-1,4-benzodiaz epin-3-yl}amino)-2-oxoethyl]-4-fluorobenzamide (enantiomers A and B)

Using aniline, the title compound was obtained as a racemate by the same method as in Example 1 (Step 1), methods equivalent thereto or combinations of these with usual methods.

Optical resolution of the racemate obtained in the above-mentioned reaction was carried out by chiral column chromatography (Daicel CHIRALPAK AD-H (20*250 mm, 5 μm), hexane:isopropyl alcohol=50:50) to yield enantiomer A (faster: 58 mg) and enantiomer B (slower: 55 mg) of the title compound as white solids, respectively.

The analytical data of the title compound are shown below.

¹H-NMR (CDCl3) δ: 1.79 (3H, s) 1.81 (3H, s), 3.26 (3H, s), 4.05 (1H, d, J=14.5 Hz), 4.16 (1H, d, J=14.5 Hz), 5.3 9 (1H, d, J=7.0 Hz), 7.02-7.16 (7H, m), 7.23 (1H, d, J=8.2 Hz), 7.26 (1H, d, J=7.0 Hz), 7.49 (1H, dd, J=7.8, 7.8 Hz), 7.57 (1H, d, J=7.4 Hz), 7.78 (1H, d, J=6.3 Hz), 7.80 (1H, d, J=8.6 Hz), 7.81 (1H, d, J=8.6 Hz).

ESI-MS (m/e): 572 [M+H]+

### Example 4

### N-[1,1-dimethyl-2-({1-methyl-2-oxo-5-[4-(trifluoromethoxy)benzyl]-2,3-dihydro-1H-1,4-benzodiaz epin-3-yl}amino)-2-oxoethyl]benzamide (enantiomers A and B)

Using 2-methyl-2-[(phenylcarbonyl)amino]propionic acid, the title compound was obtained as a racemate by the same method as in Example 3.

Optical resolution of the resultant racemate was carried out by chiral column chromatography (Daicel CHIRALPAK AD-H (20*250 mm, 5 μm), hexane:isopropyl alcohol=50:50) to yield enantiomer A (faster: 51 mg) and enantiomer B (slower: 51 mg) of the title compound as white solids, respectively.

The analytical data of the title compound are shown below.

¹H-NMR (CDCl3) δ: 1.80 (3H, s),

1.82 (3H, s), 3.25 (3H, s), 4.04 (1H, d, J=14.8 Hz), 4.15 (1H, d, J=14.8 Hz), 5.39 (1H, d, J=7.6 Hz), 7.02 (2H, d, J=8.4 Hz), 7.11 (2H, d, J=8.8 Hz), 7.17-7.27 (3H, m), 7.40 (2H, dd, J=8.0, 8.0 Hz), 7.48 (2H, dd, J=8.0, 8.0 Hz), 7.5 6 (1H, d, J=8.0 Hz), 7.79-7.84 (3H, m).

ESI-MS (m/e): 554 [M+H]+

The usefulness of the compound represented by the formula (I) as a medicament is proved, for example, in the assay described below.

### Cloning of Human DGAT1 Gene and Expression in Yeast

Human DGAT1 genes were amplified by PCR using primers described below from human cDNA library (Clontech).

| | |
|---|---|
| DGAT1F: | 5′-ATGGGCGACCGCGGCAGCTC-3′ |
| DGAT1R: | 5′-CAGGCCTCTGCCGCTGGGGCCTC-3′ |

The amplified human DGAT1 genes were introduced into a yeast expression vector pPICZA (Invitrogen). The resultant expression plasmid was introduced into an yeast (Pichia pastris) by electroporation to produce a recombinant yeast. The recombination yeast was cultured in the presence of 0.5% methanol for 72 hours, and the cells were crushed using glass beads in 10 mM Tris pH 7.5, 250 mM sucrose and 1 mM EDTA, followed by adjusting the membrane fraction by centrifugation to use the adjusted membrane fraction as an enzyme source.

### <DGAT1 Inhibitory Activity Test>

To the reaction liquid having the following composition: 100 mM Tris pH 7.5, 100 mM MgCl₂, 100 mM sucrose, 40 μM dioelin, 15 μM [¹⁴C]-oleoyl-CoA, 0.25 μg of test substance, DGAT1-expressed yeast membrane fraction, was added, and the mixture having a volume of 100 μl was incubated at room temperature for 30 minutes. To the reaction liquid, 100 μl of 2-propanol/heptan/H₂O (80:20:2) was added, the mixture was stirred well, followed by adding 200 μl of heptane and further stirring the mixture. After centrifugation, the heptane layer was collected, ethanol/0.1N NaOH/H₂O (50:5:45) was added, the mixture was thus stirred, followed by re-centrifuging the mixture and collecting the heptane layer. After exsiccation of the resultant heptane layer, 100 μl of Microscint 0 (PerkinElmer) was added, and the radioactivity was measured with a liquid scintillation counter. The inhibitory activity was calculated from the following formula:

Inhibition rate=100−(radioactivity in case of addition of test compound−background)/(radioactivity in case of addition of no test compound−background)×100 wherein the background means the radioactivity in case of addition of no membrane fraction.

The DGAT1 inhibitory activity of the compound according to the present invention by the aforementioned method is shown below.

TABLE 5

| | DGAT1 Inhibitory |
|---|---|
| | Activity |
| Example Number | IC₅₀ (nM) |
| Example 1 Enantiomer A | 483 |
| Example 1 Enantiomer B | 133 |
| Example 2 Enantiomer A | 569 |
| Example 2 Enantiomer B | 164 |
| Example 3 Enantiomer A | 181 |
| Example 3 Enantiomer B | 125 |
| Example 4 Enantiomer A | 308 |
| Example 4 Enantiomer B | 127 |

As shown in Table 5, the compound according to the present invention has excellent DGAT1 inhibitory activity in consideration of an index of IC₅₀.

1. A compound represented by a formula (I): 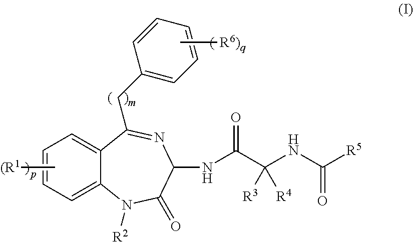
wherein R¹ each independently represents a hydrogen atom, a halogen atom or trifluoromethoxy;
R² represents lower alkyl, which may be substituted with 1 to 3 same or different halogen atoms;
R³ and R⁴ each independently represent lower alkyl;
R⁵ is a group selected from the group consisting of:
(1) phenyl, which may be substituted with 1 to 3 same or different groups selected from the group consisting of halogen atoms, lower alkoxy and trifluoromethyl;
(2) heteroaryl selected from the group consisting of pyridinyl, pyrazinyl, imidazolyl, thiazolyl, thienyl and oxazolyl, which heteroaryl may be substituted with 1 to 3 same or different halogen atoms or lower alkyl groups which may be substituted with 1 to 3 same or different halogen atoms;
(3) —O—C₃₋₆ branched lower alkyl, which may be substituted with 1 to 3 same or different halogen atoms; and
(4) C₃₋₇ cycloalkyl, which may be substituted with trifluoromethyl;
R⁶ each independently represents a group selected from the group consisting of a hydrogen atom, a halogen atom and trifluoromethoxy;
m is an integer of from 0 to 2;
p is an integer of from 1 to 4; and
q is an integer of from 1 to 5,
or a pharmaceutically acceptable salt thereof.

2. The compound according to claim 1, wherein both R³ and R⁴ are methyl, or a pharmaceutically acceptable salt thereof.

3. The compound according to claim 1, wherein R² is methyl or fluoromethyl, or a pharmaceutically acceptable salt thereof.

4. The compound according to claim 1, wherein R² is methyl, or a pharmaceutically acceptable salt thereof.

5. The compound according to claim 1,
wherein m is 1;
R² is methyl; and
both R³ and R⁴ are methyl,
or a pharmaceutically acceptable salt thereof.

6. The compound according to claim 5, wherein R⁵ is a group selected from the group consisting of:
(1-1) phenyl, which may be substituted with 1 or 2 same or different groups selected from the group consisting of fluorine and chlorine atoms, and difluoromethoxy and trifluoromethoxy;
(2-1) heteroaryl selected from the group consisting of pyridinyl and imidazolyl, which heteroaryl may be substituted with 1 or 2 same or different groups selected from the group consisting of fluorine and chlorine atoms and methyl;
(3-1) tert-butoxy or 1-ethylpropoxy; and
(4-1) 1-(trifluoromethyl)cyclopropyl or 4-trifluoromethylcyclohexyl,
or a pharmaceutically acceptable salt thereof.

7. The compound according to claim 5, wherein R⁵ is a group selected from the group consisting of phenyl, 4-chlorophenyl, 4-fluorophenyl, 3,4-difluorophenyl, 4-difluoromethoxyphenyl, 4-trifluoromethoxyphenyl, 5-fluoro-2-pyridinyl, 2-fluoro-4-pyridinyl, tert-butoxy, 1-(trifluoromethyl)cyclopropyl and 4-trifluoromethylcyclohexyl, or a pharmaceutically acceptable salt thereof.

8. The compound according to claim 5,
wherein R⁵ is a group selected from the group consisting of phenyl, 4-fluorophenyl, 4-difluoromethoxyphenyl, 4-trifluoromethoxyphenyl, 5-fluoro-2-pyridinyl, 1-(trifluoromethyl)cyclopropyl and tert-butoxy,
or a pharmaceutically acceptable salt thereof.

9. The compound according to claim 1, wherein a group represented by a formula (II): 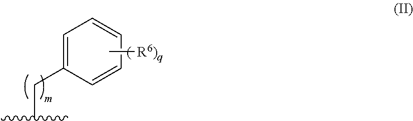
wherein [symbol]
represents a binding site, in the formula (I) is selected from the group consisting of 4-trifluoromethoxyphenyl, 3,5-dichlorophenyl, 2-chlorophenyl, 4-fluoro-3-trifluoromethylphenyl, 2-trifluoromethylphenyl, 2-fluorophenyl, 2-trifluoromethoxyphenyl, 4-trifluoromethoxyphenyl, 3,5-difluorophenyl, 3,4-dichlorophenyl, 3,5-dichlorophenyl, 4-chlorophenyl, 4-chloro-3-fluorophenyl, 3,4-difluorophenyl, 3-methylphenyl, 3-trifluoromethoxyphenyl, 3,5-bis(trifluoromethyl)phenyl and 3-chloro-5-fluorophenyl,
or a pharmaceutically acceptable salt thereof.

10. The compound according to claim 1,
wherein the compound represented by the formula (I) is 4-fluoro-N-[2-({7-fluoro-1-methyl-2-oxo-5-[4-(trifluoromethoxy)benzyl]-2,3-dihydro-1H-1,4-benzodiazepin-3-yl}amino)-1,1-dimethyl-2-oxoethyl]benzamide, N-[2-({7-fluoro-1-methyl-2-oxo-5-[4-(trifluoromethoxy)benzyl]-2,3-dihydro-1H-1,4-benzodiazepin-3-yl}amino)-1,1-dimethyl-2-oxoethyl]benzamide, N-[1,1-dimethyl-2-({1-methyl-2-oxo-5-[4-(trifluoromethoxy)benzyl]-2,3-dihydro-1H-1,4-benzodiazepin-3-yl}amino)-2-oxoethyl]-4-fluorobenzamide or N-[1,1-dimethyl-2-({1-methyl-2-oxo-5-[4-(trifluoromethoxy)benzyl]-2,3-dihydro-1H-1,4-benzodiazepin-3-yl}amino)-2-oxoethyl]benzamide,
or a pharmaceutically acceptable salt thereof.

11. A pharmaceutical composition comprising:
the compound according to claim 1; and
a pharmaceutically acceptable carrier.

12. (canceled)

13. (canceled)

14. A method for treating hyperlipidemia, diabetes and/or obesity comprising administering a therapeutically effective amount of a compound of claim 1 to a patient in need thereof.


SEQUENCE LISTING (2 sequences)

SEQ ID NO: 1 - DNA, 20 bp, homo sapiens
atgggcgaccgcggcagctc

SEQ ID NO: 2 - DNA, 23 bp, homo sapiens
caggcctctgccgctggggcctc